(19) United States
(12) Patent Application Publication
(10) Pub. No.: US 2014/0328864 A1
(43) Pub. Date: Nov. 6, 2014

(54) METHODS AND PHARMACEUTICAL COMPOSITIONS FOR REDUCING AIRWAY HYPERRESPONSE

(71) Applicants: Yann Herault, Illkirch Cedex (FR); Emilie Dalloneau, Illkirch Cedex (FR)

(72) Inventors: Yann Herault, Illkirch Cedex (FR); Emilie Dalloneau, Illkirch Cedex (FR)

(73) Assignees: INSERM (INSTITUT NATIONAL DE LA SENTE ET DE LA RECHERCHE MEDICALE), Paris (FR); UNIVERSITE DE STRASBOURG, Strasbourg (FR); CNRS (CENTRE NATIONAL DE LA RECHERCHE SCIENTIFIQUE), Paris 16 (FR)

(21) Appl. No.: 14/359,271

(22) PCT Filed: Nov. 22, 2012
(86) PCT No.: PCT/EP2012/073344

§ 371 Date: May 19, 2014

(30) Foreign Application Priority Data
Nov. 22, 2011 (EP) 11306531.2

(51) Int. Cl. C07K 16/18; G01N 33/68; C12N 15/113

(52) U.S. Cl. CPC C07K 16/18; C12N 15/113; G01N 33/6893; G01N 2333/47; G01N 2800/12
USPC 424/172.1; 514/44 A; 436/501; 435/7.92

(57) ABSTRACT

The present invention relates to agent selected from the group consisting of an anti-S100B antibody, an anti-S100B aptamer or an inhibitor of S100B gene expression for use in a method for reducing airway hyperresponse in a subject in need thereof. The present invention also relates to a method for determining whether a subject is at risk of having or developing an airway hyperresonse comprising determining the level of S100B protein in a biological sample obtained from said subject.

### FIELD OF THE INVENTION

The present invention relates to methods and pharmaceutical composition for reducing airway hyperresponse in a subject in need thereof.

### BACKGROUND OF THE INVENTION

Airway hyperresponse (“AHR”) is a characteristic feature of many airway diseases and consists of an abnormality of the airways that allows them to narrow too easily and/or too much in response to a stimulus. Respiratory diseases, associated with a variety of conditions, are extremely common in the general population. Airway hyperresponse is observed in particular in asthma and chronic obstructive pulmonary disease (COPD).

Asthma is one of the most common diseases in industrialized countries. Asthma is a condition characterized by variable, in many instances reversible obstruction of the airways. This process is associated with lung inflammation and in some cases lung allergies. Many subjects have acute episodes referred to as “asthma attacks,” while others are afflicted with a chronic condition. All asthmatics have a group of symptoms, which are characteristic of this condition: episodic bronchoconstriction, lung inflammation and decreased lung surfactant. Existing bronchodilators and antiinflammatories are currently commercially available and are prescribed for the treatment of asthma. Most of the drugs available for the treatment of asthma are, more importantly, barely effective in a small number of subjects.

COPD is characterized by airflow obstruction that is generally caused by chronic bronchitis, emphysema, or both. Commonly, the airway obstruction is incompletely reversible but 10-20% of subjects do show some improvement in airway obstruction with treatment. In chronic bronchitis, airway obstruction results from chronic and excessive secretion of abnormal airway mucus, inflammation, bronchospasm, and infection. There is very little currently available treatment to alleviate symptoms of COPD, prevent exacerbations, preserve optimal lung function, and improve daily living activities and quality of life. Many subjects will use medication chronically for the rest of their lives, with the need for increased doses and additional drugs during exacerbations. Medications that are currently prescribed for COPD subjects include: fast-acting-beta2-agonists, anticholinergic bronchodilators, long-acting bronchodilators, antibiotics, and expectorants. Amongst the currently available treatments for COPD, short term benefits, but not long term effects, were found on its progression, from administration of anti-cholinergic drugs, beta2 adrenergic agonists, and oral steroids. Short and long acting inhaled beta2 adrenergic agonists achieve short-term bronchodilation and provide some symptomatic relief in COPD subjects, but show no meaningful maintenance effect on the progression of the disease.

Thus, there is a need for additional treatment options in managing airway hyper-responsiveness in airway diseases such as asthma or COPD.

S100B is a member of the S100 protein family. S100 protein is a low molecular weight protein found in vertebrates characterized by two calcium binding sites of the helix-loophelix (“EF-hand type”) conformation. There are at least 21 different types of 5100 proteins. The name is derived from the fact that the protein is 100% soluble in ammonium sulfate at neutral pH. S100B is an acidic protein with a molecular weight of 12 kDa existing as a homodimer consisting of two beta subunits. The two monomers are configured in a twofold axis of rotation and are held together by disulfide bonds. S100B has been identified as a calcium binding protein playing a role in contraction of striated and skeletal muscle but its role in the airways hyper response has not yet been investigated.

### SUMMARY OF THE INVENTION

The present invention relates to agent selected from the group consisting of an anti-S100B antibody, an anti-S100B aptamer or an inhibitor of S100B gene expression for use in a method for reducing airway hyperresponse in a subject in need thereof. The present invention also relates to a method for determining whether a subject is at risk of having or developing an airway hyperresonse comprising determining the level of S100B protein in a biological sample obtained from said subject.

### DETAILED DESCRIPTION OF THE INVENTION

Bronchial hyperactivity is a sign of smooth cell muscle contraction, leading to constricted airways and difficulties to breath. The reactivity is induced by bacterial or allergic derivatives in mouse models of acute lung injury and of allergic asthma. The inventors have addressed the contribution of S100b, a ligand of the RAGE/AGER pathway, in the airway responses in two models of respiratory challenges. S100b was expressed in the lungs following airway stimulation with a dynamic profile similar to pro-inflammatory cytokines. Mouse lacking S100b displayed reduced LPS-induced airway response with no impact on the cellular recruitment or on the secretion of TNF-α and IL-6 pro-inflammatory cytokines. Similarly, neutralizing antibodies treatment abrogated bronchial reactivity in the LPS challenge and in the OVA-sensitized model of allergic asthma. After LPS challenge, 5100b expressing cells accumulated near the upper airway epithelial and muscular cells in the bronchiole, suggesting a potential role of S100B in controlling the dynamic of cellular remodeling during the airway hyperresponsiveness. These observations highlighted S100b upstream of the cascade regulating bronchial hyperreactivity and showed that S100B blockade as a potential therapeutic strategy for respiratory diseases including acute lung injury and asthma.

Accordingly the present invention relates to an agent selected from the group consisting of an anti-S100B antibody, an anti-S100B aptamer or an inhibitor of S100B gene expression for use in a method for reducing airway hyperresponse (AHR) in a subject in need thereof.

It is worth mentioning that the agents of the invention are able to reduce AHR without modifying the inflammatory response (see Example 1).

As used herein, the term “S100B” has its general meaning in the art and refers to S100 calcium binding protein B described in Donato R. S100: a multigenic family of calcium-modulated proteins of the EF-hand type with intracellular and extracellular functional roles. Int J Biochem Cell Biol. 2001 July; 33(7):637-68.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or non human antibody. A non human antibody may be humanized by recombinant methods to reduce its immunogenicity in man.

As used herein the term “anti-S100B antibody” refers to any antibody directed against S100B.

Antibodies may be prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of S100B. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, recombinant forms of S100B may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods. Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope. The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated a Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

As used herein the term “aptamer” refers to a a class of molecule that represents an alternative to antibodies in term of molecular recognition. Aptamers are oligonucleotide or oligopeptide sequences with the capacity to recognize virtually any class of target molecules with high affinity and specificity. Accordingly the term “anti-S100B aptamer” refers to an apatamer directed against S100B.

Aptamers may be isolated through Systematic Evolution of Ligands by EXponential enrichment (SELEX) of a random sequence library. The random sequence library is obtainable by combinatorial chemical synthesis of DNA.

An “inhibitor of S100B gene expression” refers to a natural or synthetic compound that has a biological effect to inhibit or significantly reduce the expression of S100B gene.

Inhibitors of expression for use in the present invention may be based on anti-sense oligonucleotide constructs. Anti-sense oligonucleotides, including anti-sense RNA molecules and anti-sense DNA molecules, would act to directly block the translation of S100B mRNA by binding thereto and thus preventing protein translation or increasing mRNA degradation, thus decreasing the level of S100B, and thus activity, in a cell. For example, antisense oligonucleotides of at least about 15 bases and complementary to unique regions of the mRNA transcript sequence encoding S100B can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of expression for use in the present invention. S100B gene expression can be reduced by contacting a subject or cell with a small double stranded RNA (dsRNA), or a vector or construct causing the production of a small double stranded RNA, such that S100B gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). All or part of the phosphodiester bonds of the siRNAs of the invention are advantageously protected. This protection is generally implemented via the chemical route using methods that are known by art. The phosphodiester bonds can be protected, for example, by a thiol or amine functional group or by a phenyl group. The 5′- and/or 3′-ends of the siRNAs of the invention are also advantageously protected, for example, using the technique described above for protecting the phosphodiester bonds. The siRNAs sequences advantageously comprise at least twelve contiguous dinucleotides or their derivatives.

As used herein, the term “siRNA derivatives” with respect to the present nucleic acid sequences refers to a nucleic acid having a percentage of identity of at least 90% with erythropoietin or fragment thereof, preferably of at least 95%, as an example of at least 98%, and more preferably of at least 98%.

As used herein, “percentage of identity” between two nucleic acid sequences, means the percentage of identical nucleic acid, between the two sequences to be compared, obtained with the best alignment of said sequences, this percentage being purely statistical and the differences between these two sequences being randomly spread over the nucleic acid acids sequences. As used herein, “best alignment” or “optimal alignment”, means the alignment for which the determined percentage of identity (see below) is the highest. Sequences comparison between two nucleic acids sequences are usually realized by comparing these sequences that have been previously align according to the best alignment; this comparison is realized on segments of comparison in order to identify and compared the local regions of similarity. The best sequences alignment to perform comparison can be realized, beside by a manual way, by using the global homology algorithm developed by SMITH and WATERMAN (Ad. App. Math., vol. 2, p:482, 1981), by using the local homology algorithm developed by NEDDLEMAN and WUNSCH (J. Mol. Biol., vol. 48, p:443, 1970), by using the method of similarities developed by PEARSON and LIPMAN (Proc. Natl. Acd. Sci. USA, vol. 85, p:2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C., Nucleic Acids Research, vol. 32, p:1792, 2004). To get the best local alignment, one can preferably used BLAST software. The identity percentage between two sequences of nucleic acids is determined by comparing these two sequences optimally aligned, the nucleic acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

shRNAs (short hairpin RNA) can also function as inhibitors of expression for use in the present invention.

Ribozymes can also function as inhibitors of expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of S100B mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or desoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and preferably cells expressing S100B. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adenoviruses and adeno-associated (AAV) viruses, which are double-stranded DNA viruses that have already been approved for human use in gene therapy. Actually 12 different AAV serotypes (AAV1 to 12) are known, each with different tissue tropisms (Wu, Z Mol Ther 2006; 14:316-27). Recombinants AAV are derived from the dependent parvovirus AAV2 (Choi, V W J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

According to the present invention, “airway hyperresponse” or “AHR” refers to an abnormality of the airways that consists in an exaggerated airway-narrowing response to many environmental triggers, such as allergen and exercise. AHR can be a functional alteration of the respiratory system caused by inflammation or airway remodeling. Airway hyperresponse can be caused by collagen deposition, bronchospasm, airway smooth muscle hypertrophy, airway smooth muscle contraction, mucous secretion, cellular deposits, epithelial destruction, alteration to epithelial permeability, alterations to smooth muscle function or sensitivity, abnormalities of the lung parenchyma and/or infiltrative diseases in and around the airways. Many of these causative factors can be associated with inflammation. The present invention is directed to any airway hyperresponse, including airway hyperresponse that is associated with inflammation of the airway (e.g. eosinophilia and inflammatory cytokine production).

As used herein, “reducing airway hyperresponse” refers to any measurable reduction in airway hyperresponse and/or any reduction of the occurrence or frequency with which airway hyperresponse occurs in a subject. Preferably, airway hyperresponse is reduced, optimally, to an extent that the subject no longer suffers discomfort and/or altered function resulting from or associated with airway hyperresponse. A reduction in AHR can be measured using any suitable method known in the art. Respiratory function can be measured by, for example, spirometry, plethysmography, peak flows, symptom scores, physical signs (i.e., respiratory rate), wheezing, exercise tolerance, use of rescue medication (i.e., bronchodialators) and blood gases.

In a particular embodiment airway hyperresponse is associated with allergic inflammation.

In a particular embodiment, the subject suffers from a airway disease selected from the group consisting of asthma, chronic obstructive pulmonary disease, allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma, sarcoid, reactive airway disease syndrome, interstitial lung disease, hyper-eosinophilic syndrome, rhinitis, sinusitis, exercise-induced asthma, pollution induced asthma and parasitic lung disease. Preferably the subject suffers from asthma, chronic obstructive disease of the airways, occupational asthma, exercise-induced asthma, pollution-induced asthma and reactive airway disease syndrome, with chronic obstructive disease of the airways. The subject may also suffer from an airway hyperresponse associated with viral infection such as infections caused by respiratory syncytial virus (RSV), parainfluenza virus (PIV), rhinovirus (RV) or adenovirus.

The agent of the invention may be administered in the form of a pharmaceutical composition, as defined below. Preferably, said agent in a therapeutically effective amount. By a “therapeutically effective amount” is meant a sufficient amount of the agent to treat AHR at a reasonable benefit/risk ratio applicable to any medical treatment.

It will be understood that the total daily usage of the agent or pharmaceutical composition comprising thereof will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific agent employed; the duration of the treatment; drugs used in combination or coincidental with the specific agent employed; and like factors well known in the medical arts. For example, it is well within the skill of the art to start doses of the agent at levels lower than those required to achieve the desired therapeutic effect and to gradually increase the dosage until the desired effect is achieved. However, the daily dosage of the agent may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the agent for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The agent of the invention can thus be formulated into pharmaceutical compositions that further comprise a pharmaceutically acceptable carrier, diluent, adjuvant or vehicle. In one embodiment, the present invention relates to a pharmaceutical composition comprising an agent of the invention described above, and a pharmaceutically acceptable carrier, diluent, adjuvant or vehicle. In one embodiment, the present invention is a pharmaceutical composition comprising an effective amount of an agent of the present invention or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

A pharmaceutically acceptable carrier may contain inert ingredients which do not unduly inhibit the biological activity of the agent. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, its use is contemplated to be within the scope of this invention.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compositions described herein may be administered orally, parenterally, by inhalation spray, topically, rectally, nasally, buccally, vaginally or via an implanted reservoir depending on the severity of the airway hyperresponse being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a nontoxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the agent is combined with emulsifying and suspending agents. If desired, certain sweetening, flavoring or coloring agents may also be added.

Alternatively, the pharmaceutical compositions described herein may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions may also be administered to the respiratory tract. Pulmonary delivery compositions can be delivered by inhalation by the subject of a dispersion so that the agent within the dispersion can reach the lung where it can, for example, be readily absorbed through the alveolar region directly into blood circulation. Pulmonary delivery can be achieved by different approaches, including the use of nebulized, aerosolized, micellular and dry powder-based formulations; administration by inhalation may be oral and/or nasal. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self contained. Dry powder dispersion devices, for example, deliver drugs that may be readily formulated as dry powders. A pharmaceutical composition of the invention may be stably stored as lyophilized or spray-dried powders by itself or in combination with suitable powder carriers. The delivery of a pharmaceutical composition of the invention for inhalation can be mediated by a dosing timing element which can include a timer, a dose counter, time measuring device, or a time indicator which when incorporated into the device enables dose tracking, compliance monitoring, and/or dose triggering to a subject during administration of the aerosol medicament. Examples of pharmaceutical devices for aerosol delivery include metered dose inhalers (MDIs), dry powder inhalers (DPIs), and air-jet nebulizers.

A further object of the invention relates to a method for determining whether a subject is at risk of having or developing an airway hyperresponse comprising determining the level of S100B protein in a biological sample obtained from said subject.

As used herein the term biological sample encompasses any sample obtained from the patient for the purpose of determining whether a subject is at risk of having or developing an airway hyperresonse. Typically, said biological may be a blood sample or bronchoalveolar lavage fluid sample.

Determining the level of S100B in the biological sample may be performed by a variety of techniques. Typically, determining the level comprises contacting the sample with a binding partner directed against S100B, and thereby detecting the presence, or measuring the amount, of S100B in the seminal sample. As used herein, the term “binding partner” refers to any molecule (natural or not) that is able to bind the biomarker with high affinity. Said binding partners include but are not limited to antibodies or aptamers.

The binding partners of the invention such as antibodies or aptamers may be labelled with a detectable molecule or substance, such as preferentially a fluorescent molecule, or a radioactive molecule or any others labels known in the art. Labels are known in the art that generally provide (either directly or indirectly) a signal. As used herein, the term “labelled”, with regard to the antibody or aptamer, is intended to encompass direct labelling of the antibody or aptamer by coupling (i.e., physically linking) a detectable substance, such as a fluorophore [e.g. fluorescein isothiocyanate (FITC) or phycoerythrin (PE) or Indocyanine (Cy5)]) or a radioactive agent to the antibody or aptamer, as well as indirect labelling of the probe or antibody by reactivity with a detectable substance. An antibody or aptamer of the invention may be labelled with a radioactive molecule by any method known in the art. For example radioactive molecules include but are not limited radioactive atom for scintigraphic studies such as I123, I124, In111, Re186, Re188.

Contacting may be performed in any suitable device, such as a plate, microtiter dish, test tube, well, glass, column, and so forth. In specific embodiments, the contacting is performed on a substrate coated with the binding partner. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as an antibody-antigen complex, to be formed between the binding partner and the biomarker of the sample.

The presence of S100B can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

The methods of the invention may further comprise a step consisting of comparing the expression level of S100B with a reference value, wherein detecting differential in the expression of the level determined in the sample and the reference value is indicative whether the subject is at risk for having or developing airway hyperresponse.

In one embodiment, the reference value may be index values or may be derived from one or more risk prediction algorithms or computed indices for airway hyperresponse event. A reference value can be relative to a number or value derived from population studies, including without limitation, such subjects having similar body mass index, subjects of the same or similar age range, subjects in the same or similar ethnic group, subjects having family histories of airway diseases, or relative to the starting sample of a subject undergoing treatment for airway disease that can give rise to airway hyperresponse. In one embodiment of the present invention, the reference value is derived from the level of S100B in a control sample derived from one or more subjects who are substantially healthy (i.e. subject with no airway hyper response). Such subjects who are substantially healthy lack traditional risk factors for airway hyperresponse: for example, non-current smoker, no history of diagnosed airway disease. In another embodiment, such subjects are monitored and/or periodically retested for a diagnostically relevant period of time (“longitudinal studies”) following such test to verify continued absence of airway hyper response events. Such period of time may be one year, two years, two to five years, five years, five to ten years, ten years, or ten or more years from the initial testing date for determination of the reference value. Furthermore, retrospective measurement of S100B levels in properly banked historical subject samples may be used in establishing these reference values, thus shortening the study time required, presuming the subjects have been appropriately followed during the intervening period through the intended horizon of the product claim. Typically, the level of S100B in a subject who is at risk for airway hyperresponse is deemed to be higher than the reference value obtained from the general population or from healthy subjects.

The invention will be further illustrated by the following figures and examples. However, these examples and figures should not be interpreted in any way as limiting the scope of the present invention.

### FIGURES

FIG. 1: kinetics of apparition of S100B protein in lung and sera after LPS instillation in wt mice. Groups of 5 mice were instillated with LPS (10 μg) and euthanatized at different time points (0, 30 min, 2 h, 4 h and 6 h). Apparition of inflammation was controlled by measuring the neutrophils recruitment (A), IL6, TNF-α and S100b concentration (μg/ml) in lung (B) and sera (C). LPS induced a production of S100B in lung until 30 min (C) and in sera until 4 h. All the results were expressed by mean±sem.

FIG. 2: Evaluation of the airway hyper response in S100b^{+/-} and S100b^{-/-} mice. Intranasal administration of the control saline (NaCl) solution did not affect the PenH in wt neither in S100b^{+/-} (A) and S100b^{-/-} (B). The experiment was done twice with 5 mice for each condition. The loss of one copy of S100B gene tended to decrease PenH values (A) whereas these values were significantly decreased in S100b^{-/-} mice (B). The lung inflammation was measured by the recruitment of neutrophils (C), IL6 and TNF-α cytokines (D) and the myeloperoxidase activity (MPO) in BALFs. NaCl did not affect any of the parameters. LPS cellular and inflammatory responses did not showed any modifications for the measured parameters in heterozygote or homozygote mutants compared to wt.

FIG. 3: Treatment with S100B-antibody reduced LPS dependent airway hyper response. Groups of 5 mice treated with control isotonic NaCl solution, LPS or LPS plus 1 or 2 mg of S100B antibody. The experiment was done twice on C57BL/6J mice. 1 mg of S100B antibody was sufficient to decrease PenH values (A) but the response was more homogenous with 2 mg. The presence of the S100B antibody did not modify the neutrophils recruitment (B), MPO activity (C) or TNF-α (D) and IL-6 (E) concentration in BALFs 24H after LPS stimulation.

FIG. 4: Characterization of the S100B antibody effect on Prmt2^{+/-} mice, a model of exaggerated LPS response. (A) Prmt2^{+/-} mice instillated with LPS showed an exacerbated AHR, compared with control mice treated with an isotonic saline solution (NaCl). The response was significantly decreased with administration of 2 mg of S100B antibody. The Prmt2^{+/-} mice had an increased inflammation after LPS instillation characterized by an increase in neutrophils recruitment (B), in MPO activity (C) as well as in TNF-α (D) and IL-6 (E) concentration in BALFs, compared to wt mice. The administration of S100B antibody did not modify the increased lung inflammatory response suggesting an independent role of Prmt2 and S100B in the regulation of lung inflammation. All the results were expressed by mean±sem. Student's t-test: *P≦0.05.

FIG. 5: Expression of S100b in the Ms1Yah mouse model. (A) The concentration of S100b was found lower in the BALF 90m min after systemic injection of LPS in control and Ms1Yah mutant mice compared to control mice. No change was observed after saline control solution (NaCl) injection (A) or in plasma serum (B). The expression of S100b was increased in the lung of Prmt2 mutant mice 2 h after LPS stimulation compared to Ms1Yah (C). Student's t-test: **P≦0.01.

FIG. 6: S100b neutralizing antibody reduced lung response in the OVA model. (A) The recruitment of eosinophils was observed after OVA sensitized BALB/cJ mice compared to control mice (groups of n=6 individuals; repeated twice). (B) S100b concentration increased in the serum of BALB/cJ after the last OVA sensitization and Methacholine (Mch) enhanced the production of S100B 24 hours after the last ovalbumin injection. No respiratory effect was found if the mice are not sensitized to OVA and treated 16 h before Mch with the neutralizing antibody. On the contrary the neutralizing antibody injected 16 h before the last OVA sensitization decreased significantly the Penh values in treated mice compared to non-treated. Student's t-test: *P≦0.05.

FIG. 7: immunohistostaining of S100b protein on hematoxylin stained lung sections from wt mice 4 h after NaCl (A, B, D) or LPS treatment (C,E) (original magnification: A: x40; B-E: x20). (A) Rare cells, probably resident alveolar macrophages (A), were S100b positive in non-stimulated lung. (B-E) Number of cells expressing S100B was clearly increased after LPS instillation in the alveoli (B-C) and around the bronchioles (D,E).

### EXAMPLE 1

### The S100 Calcium Binding Protein B Regulates Airway Responses in LPS-Induced Acute Lung Injury and Ovalbumin-Induced Asthma Models

Introduction:

The prevalence of respiratory diseases (RD), such as asthma, has increased over the last decades (Umetsu et al., 2002). To better understand the pathophysiology, identify key markers and test therapeutic strategies, models of RD have been developed in mice (Matute-Bello et al., 2008; Nials and Uddin, 2008). As such, the instillation of lipopolysaccharides (LPS) induced pulmonary response in mice, similar to acute lung injury, and the sensitization to ovalbumine (OVA) followed by metacholine stimulation is currently used to model asthma. Those RD models trigger the inflammatory and the immune responses, with specific actions on the lung epithelial cells.

After LPS stimulation, the airway displayed a hyperresponse that can be monitored by non-invasive plethymography. Macrophages and neutrophils are recruited and activated in the airways and pro-inflammatory cytokines, such as TNF-α and IL-6, are produced locally. Briefly, LPS, associated or not with additional molecules, binds to its receptor, TLR4, that leads to the activation of the NF-κB transcription factor which translocates to the nucleus where it stimulates the transcription of genes (Dalloneau et al., 2011a; Karin and Ben-Neriah, 2000; Takeda and Akira, 2007). Beside the classical pathway, LPS induces an increase of the expression of AGER, the “Advanced Glycosylation End product-specific Receptor” (also called RAGE), in the broncho-alveolar space (Uchida, 2006) and of its ligand the S100 calcium binding protein B, S100b (Morbini et al., 2006). AGER belongs to the superfamily of immunoglobulins of cell surface receptor. AGER interacts with the TLR signaling pathways through TIRAP and MyD88 to regulate inflammatory responses and other functions (Sakaguchi et al., 2011b). AGER is expressed in a constitutive way during the development, and then its expression is restricted in certain tissues at the adulthood (Brett et al., 1993; Sasaki et al., 2001). However, its expression is more important in the lung where it is found in the type I and II alveolar pneumocytes, in macrophages and in the bronchoepithelial cells (Cheng et al., 2005; Dahlin, 2004; Morbini et al., 2006).

S100b is one of the AGER ligands and belongs to a family of 25 proteins with Ca²⁺-binding properties (Donato, 2001; Donato et al., 2009). Like other members of the family, S100b encompasses two Ca²⁺-binding sites of the EF-hand type, interconnected by a hinge region, and a C-terminal region (Donato, 1999). S100b exists either as a homodimer (Rustandi et al., 2000) or as an S100b/S100a1 heterodimer (Donato, 2001). S100b is expressed in a restricted number of cell types with different outcomes depending on the cell type and the microenvironment. Intracellular S100b can alter cell proliferation and differentiation (Arcuri, 2004), modulates the microtubule assembly (Donato, 1988), regulates the p53 dependent transcription (Wilder et al., 2006) or inhibits apoptosis and differentiation (Donato et al., 2009). In addition S100b is released by astrocytes into the extracellular space (Eldik and Zimmer, 1987) and it is also found in the serum (Donato et al., 2009). As an extracellular factor, S100b interacts with AGER and leads to beneficial or detrimental outcomes depending on the concentration of the protein, the cell type and the microenvironment (Donato et al., 2009). In the human lungs, S100b is expressed, at the level of the peri-bronchial nerves and interstitial dendritic cells (Morbini et al., 2006). The binding of S100b to AGER activates the endothelial cells and muscular lung cells, the monocytes as well as the lymphocytes T, which involves the production of the cytokines and the molecules of pro-inflammatory adhesion (Donato et al., 2009; Hofmann et al., 1999; Yan et al., 2003).

In this study, we continued further the analysis of the Ms1Yah mice that carries a deletion of the Col6a1-Prmt2 genetic interval and displays a stronger inflammatory response and an inhibited airway hyperresponse (AHR) after LPS stimulation (Besson et al., 2007). We demonstrated, the role of Prmt2 in the exaggerated inflammatory response and AHR which is a response stronger and opposite to the Ms1Yah LPS-induced phenotype (Dalloneau et al., 2011a). We found a new candidate with S100b produced in lung and sera after LPS instillation in mice. Thus we investigated the consequences of altering the S100b function, either by using knock-out approach or with neutralizing antibodies, and we found that S100b was a key player in controlling the LPS-induced AHR. In addition we demonstrated that the neutralizing antibody treatment reduced metacholine-induced airway reactivity in the OVA sensitization model of asthma. These observations put S100b as a main contributor of the cascade regulating bronchial hyperreactivity and highlighted S100b blockade as a potential therapeutic strategy for treating RD including acute lung injury and asthma.

Material and Methods:

Mouse Strains

The SMOB^{+/-} mice were described by Xiong et al. (44) carrying a deletion of the Exon 2 of S100b (which includes the ATG translation initiation codon) and its flanking 5′ and 3′ intron sequences which were replaced with a neomycin resistance (neo) selection cassette. The presence of the wild type and mutant S100b allele were identified by PCR in standard conditions with two pairs of primers. The Prmt2 and Ms1Yah mutant mice have been described previously (Besson et al., 2007; Dalloneau et al., 2011b; Yoshimoto et al., 2006).

Endotoxic Shock Induced by Administration of LPS

Animals (n=10) were treated by intranasal instillation with either isotonic saline solution or 10 μg of LPS (Escherichia coli, serotype O55B5, 10 mg, Sigma-Aldrich, St Louis, USA) in deep anaesthesia. 1 mg or 2 mg of polyclonal anti-S100B antibody (Santa cruz) were administrated by i.p. 16 h before the LPS instillation.

Airways Resistance and Broncho-Alveolar Lavage Fluids

Animals (n=10) were treated by intranasal instillation with either isotonic saline solution (NaCl 0.9%) or 10 μg of LPS (Escherichia coli, serotype 055B5, 10 mg, Sigma-Aldrich, St Louis, USA) in deep anaesthesia. Airway response was investigated over a period of 6 h after treatment using whole-body plethysmography and measurements of the PenH, a dimensionless parameter that accounts for the respiratory profile, taken in consideration the period of expiration and the variations of pressure measured in a closed chamber during the respiratory cycle (EMKA Technologies, Paris, France). PenH can be conceptualized as the phase shift of the thoracic flow and the nasal flow curves. Increased phase shift correlates with increased respiratory system resistance. PenH is calculated by the formula PenH=(Te/RT−1)×PEF/PIF, where Te is expiratory time, RT is relaxation time, PEF is peak expiratory flow, and PIF is peak inspiratory flow (Togbe et al., 2007). Data are analyzed using Datanalyst software (EMKA Technologies) and expressed as mean±sem (standard error of the mean). Twenty-four hours after stimulation, mice were euthanized and BALFs were analyzed for cell composition and cytokines quantification as described previously (Besson et al., 2007). An aliquot was stained with Trypan Blue solution and analyzed to determine cellular content. After centrifugation on microscopic slides, air-dried preparations were fixed and stained with Diff-Quick (Merz & Dade) using a May-Grünwald-Giemsa coloration. Two hundred cells were counted twice for the determination of the differential counts of each cell type in the BALF. Part of the lung was stored at −80C° for the myeloperoxidase (MPO) assay (Besson et al., 2007; Dalloneau et al., 2011a).

Measurement of Cytokines, and Myeloperoxidase Assay

IL-6 and TNF-α concentration were estimated by Elisa Test, in standard condition with the protocol of the supplier (RnD Systems). The sera were diluted at ¼ and the BALFs were used undiluted. The 96-wells plate was read by the reader EL 800 (BIO-TEK INSTRUMENTS). For the MPO measurement, the right heart ventricle was perfused with saline to flush the vascular content, and lungs were frozen at −80° C. until use. Lung was homogenized by polytron and centrifuged, and the supernatant was discarded. The pellets were resuspended in 1 ml of PBS containing 0.5% hexadecyltrimethyl ammonium bromide (HTAB) and 5 mM EDTA. After centrifugation, 50 μl of supernatants were placed in test tubes with 200 μl of PBS-HTABEDTA, 2 ml HBSS, 100 μl of o-dianisidine dihydrochloride (1.25 mg/ml), and 100 μl of H2O2 0.05%. After 15 min of incubation at 37° C. in an agitator, the reaction was stopped with 100 μl of NaN3 1%. The MPO activity was determined as absorbance at 460 nm against medium.

Systemic Endotoxic Shock Induced by Administration of LPS

Mice received an intra-peritoneal injection of 100 μg of LPS (serotype 055B5) (n=10) or saline control solution (n=6) to evaluate the systemic inflammatory response in vivo (Besson et al., 2007). After 90 min, mice were euthanized, the blood was collected through the femoral vein and centrifuged at 2000 rpm for 15 min and serum was collected and stored at −20° C. for cytokines assays. Then, the chest is opened and the lung is washed by injecting 10 ml of PBS into the right ventricle of the heart. The lungs fade to white. The heart-lung block is then removed, then the lungs were isolated and stored in a tube immersed in liquid nitrogen. The lungs were used to study the expression of target genes by QRT-PCR.

OVA Sensitization and Lung Response

We developed an acute murine model for asthma. Male BALB/cJ mice were sensitized on days 1 and 7 by intra-peritoneal injection of 100 μl of a solution composed by 0.5 mg/ml of OVA (ovalbumine, OVA, grade V-Sigma) adsorbed in 20 mg/ml of aluminum hydroxide (Sigma A8222, Sigma-Aldrich) in sterile NaCl 0.9%. Then mice were challenged to ovalbumine at days 18 to 21. For that, mice were anesthetized with ketamine (50 mg/kg) and xylazine (3.5 mg/kg) and were instillated with 25 μl of a solution of 0.4 mg/ml of OVA. On day 17, 16 h before the first challenge, a group of mice received 2 mg of anti-S100B antibody (Santa Cruz) by i.p. The control group was sensitized and challenged with NaCl 0.9% only. The bronchial reactivity was assessed by whole body plethysmography on day 22, between 18 and 24 h after the last challenge, as described above. The sera, BALFs and lungs samples were collected on day 22, just after the plethysmography acquisition. Experiment was done twice for a total number of 12 animals per groups. The protocol for the acquisition on the plethysmograph consists to 30 min of stabilization of the mice (without recording of the values), then 30 min of measurement of the basal values (recorded, called basal), then a nebulisation of 30 sec of NaCl 0.9% followed by 30 min of record of the signals, then four nebulisation of 30 seconds of increasing amount of methacholin (0.05; 0.1; 0.2 and 0.3M) separated by 20 min of record of the signal. Data are then processed with Datanalyst software.

Immunodetection of S100b

For immunodetection of S100B, α-actin and SV2, lungs were fixed by immersion in ice-cold 4% (w/v) PFA in PBS during 6 hours, washed in PBS and embedded in paraffin. Prior to immunostaining, the histological sections from paraffin-embedded tissues were collected on glass slides, treated with Tris-EDTA Buffer (10 mM Tris Base, 1 mM EDTA Solution, 0.05% Tween 20, pH 9.0) in a bath at 94° C. during 40 minutes. For immunostaining, the sections were incubated overnight at 4° C. with the rabbit polyclonal antibody S100B (dilution 1/10 000, HPA015768, Sigma Aldrich) alone for chromogen immunostaining or either combined with the mouse monoclonal antibodies SV2 (dilution 1/500, SV2-c, Developmental Studies Hybridoma Bank) or actin, a smooth muscle (dilution 1/10 000, A 5228, Sigma Aldrich) for double immunofluorescence stainings. For chromogen immunostaining of S100b, detection of the primary antibody was achieved using the elite vectastain ABC technique (PK-6101, Vector), according to manufacturer's instructions. Peroxydase was revealed using diaminobenzidine tablets (D4168, Sigma Aldrich). For immunofluorescence, detection of primary antibody was achieved by incubating the sections for 1 hour at room temperature using a Cy3-conjugated goat anti-rabbit IgG (Jackson Immuno Research Laboratories) (for S100B) or Alexa-Fluor-488-conjugated mouse antibody (Molecular Probes; for SV2 and actin) diluted at 1/500. The sections were counterstained with DAPI.

Statistical Analysis

Statistical analysis was performed using either the parametric Fischer Student's t-test when applicable or the non-parametric Wilcoxon Mann-Whitney's U test via the Statgraphics software (Centurion XV, Sigma plus, Levallois Perret). Values are presented as mean±SEM and the significant threshold was P<0.05 or otherwise indicated.

Study Approval

All mouse procedures were approved by the local ethical committee of the ICS or the TAAM. YH, as the principal investigator in this study, was granted the accreditation 45-31 and 67-369 to perform the reported experiments.

Results:

S100b is Expressed in Lung and Sera after LPS Stimulation

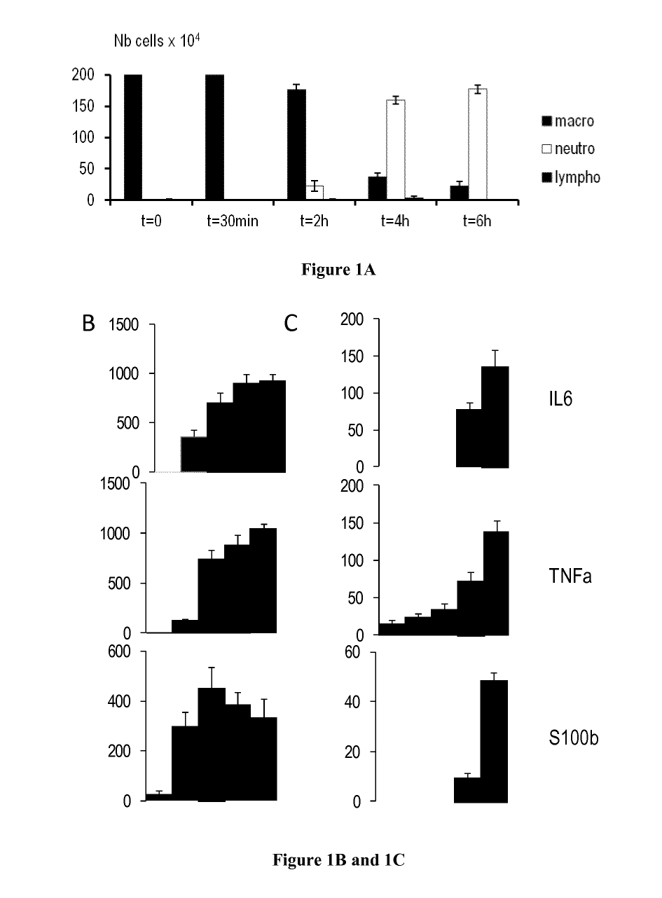

To investigate the role of S100b in the LPS-induced respiratory and inflammatory responses, we first checked the production of the protein after LPS challenge. Groups of C57BL/6J (B6) mice were instillated with LPS (10 μg) and euthanized at different time points (0, 30 min, 2 h, 4 h, and 6 h). We evaluated the lung inflammation by measuring the number of macrophages, neutrophils, and lymphocytes, plus the concentration of two pro-inflammatory cytokines, TNF-α and IL-6, in the broncho-alveolar lavages fluids (BALFs; FIG. 1). Between 0 and 2 h macrophages were the main cell type found in BALFs. Neutrophils started to be recruited after 2 h and we observed a shift with a decrease of macrophages and an increase of neutrophils between 2 h and 4 h (FIG. 1A). TNF-α was already detected at 0 and 30 min at weak concentration and was strongly produced until 2 h after the LPS challenge (FIG. 1B). Secretion of IL-6 started at 30 min with an important increase at 2 h (FIG. 1B). S100b was strongly produced in BALFs at 30 min after the LPS stimulation, showing a pick of accumulation at 2 h followed by a slow decrease after LPS treatment (FIG. 1). We observed the same phenomenon in sera where TNF-α was already present at low level at 0 min. The serum concentration of TNF-α, IL-6 and 5100b increased in a sensitive way 4 h after the LPS stimulation (FIG. 1C). All these results showed that S100B displayed the same kinetics as pro-inflammatory cytokines in both lung and blood serum. Thus S100B is a marker of the lung response with a kinetic of accumulation quite similar to pro-inflammatory cytokines directly controlled by LPS.

LPS Instillation LED to a Reduced AHR in S100b^{-/-} Mice without Affecting the Inflammation in Lung

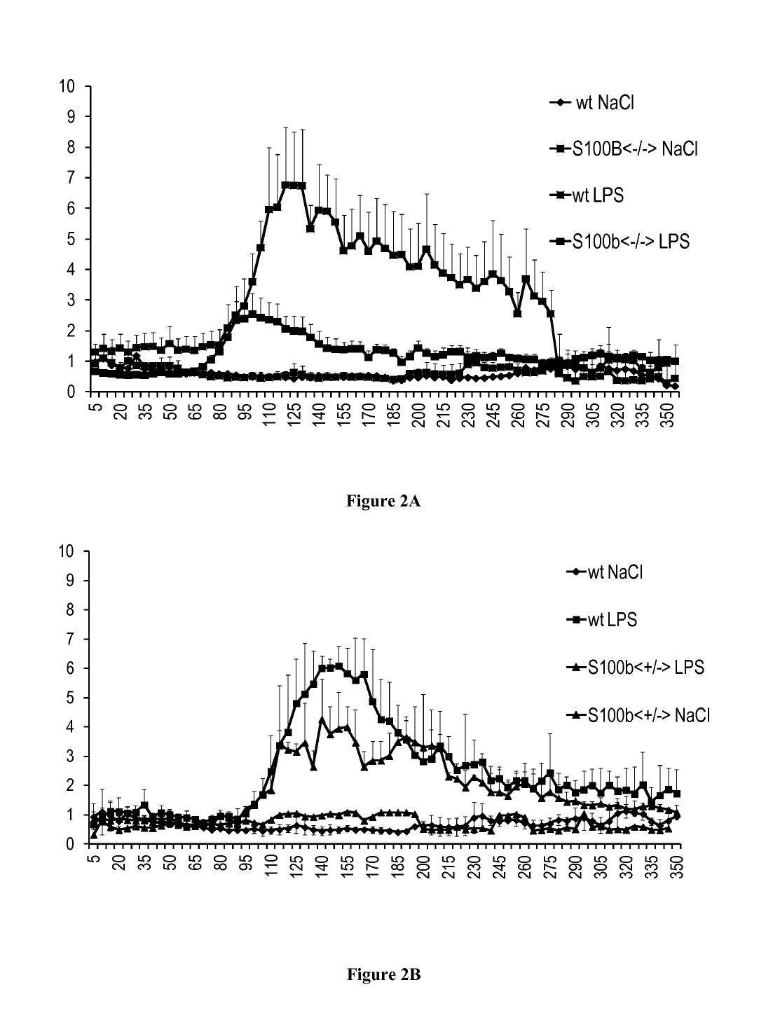

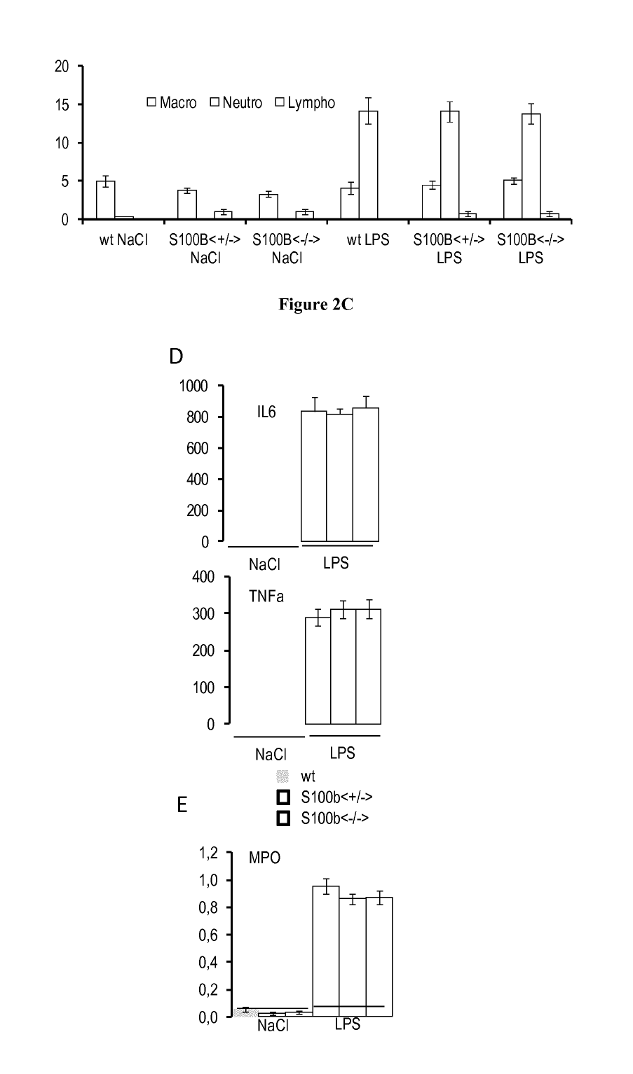

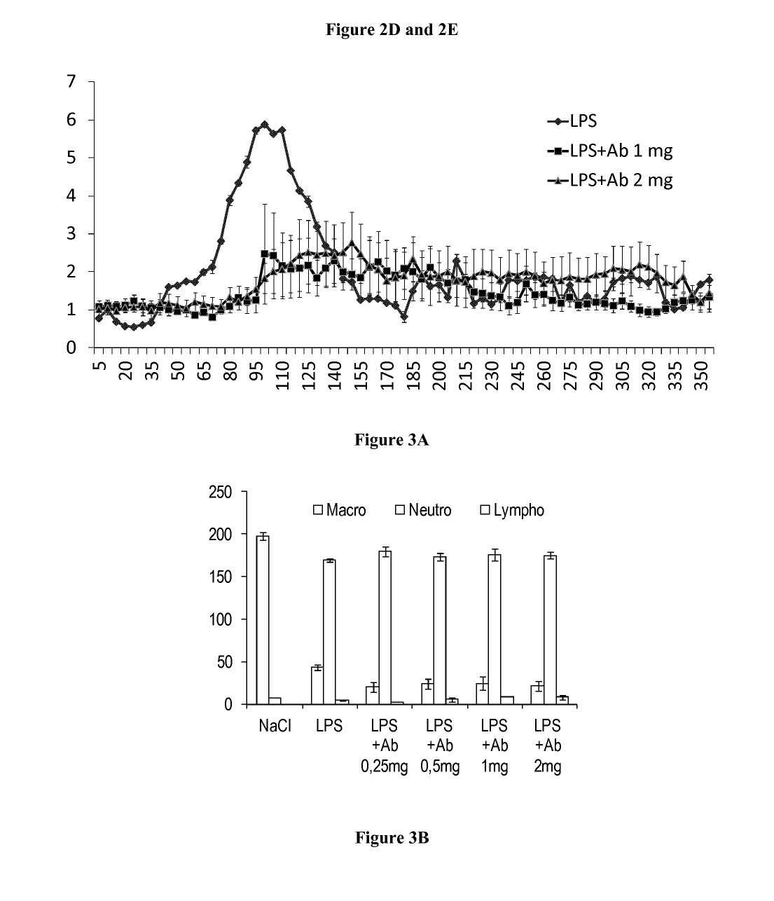

According to the accumulation of S100b in the BALF, we monitored changes in the lung following LPS challenge in S100b mutant mice. We used whole body plethysmography, and we scored the parameter of pause enhanced time (PenH) that reflects the respiratory pattern to evaluate lung response to LPS in S100b mutant mice. As shown previously Ms1Yah mice carrying a single copy of S100b have a reduced respiratory response after LPS instillation (Besson et al., 2007). To investigate the precise role of S100b in the AHR, we used a loss-of-function allele of S100b (Xiong et al., 2000) and we compared control wild-type (wt) animal, heterozygote (S100b^{+/-}) and homozygote (S100b^{-/-}) mutant individuals instillated with LPS (10 μg) or saline (NaCl) control solution. Treatment with saline solution did not affect the respiratory function as assessed by PenH in wt mice and both S100b^{+/-} and S100b^{-/-} (FIGS. 2 A-B). The instillation of LPS induced an AHR characterized by an increase in the PenH values superior to 5 in wt mice. The PenH values tended to decrease in S100b^{+/-} (FIG. 2B) whereas the AHR was significantly decreased in S100b^{-/-} mice (FIG. 2A). The loss of the two copies of S100b had a major impact on the AHR induced by LPS stimulation.

Ms1Yah mice instillated by LPS have an enhanced recruitment of inflammatory cells and production of TNF-α and IL-6 in the broncho-alveolar space in comparison with wild-type mice (Besson et al., 2007) that we correlated with Prmt2 (Dalloneau et al., 2011a). We investigated the involvement of S100b in the local inflammatory response. 24 h hours after the instillation of LPS or saline control solution, we measured the inflammation induced by LPS in wild-type, S100b^{+/-} and S100b^{-/-} mice. We followed the neutrophils recruitment (FIG. 2C), myeloperoxidase (MPO) activity (FIG. 2D) and the concentration of TNF-α and IL-6 (FIG. 2E) in BALFs. The mice instillated with the saline control solution did not present any changes in the type of inflammatory cells recruitment. The measured inflammatory parameters do not present any changes between wt and both S100b^{+/-} or S100b^{-/-} mice. Thus we demonstrated here that altering the S100b gene function strongly impaired the AHR induced by LPS but did not interfere with the inflammatory response in lung.

Neutralizing Anti-S100b Antibody Reduced AHR in C57BL/6J Mice without Affecting the Inflammation in Lung

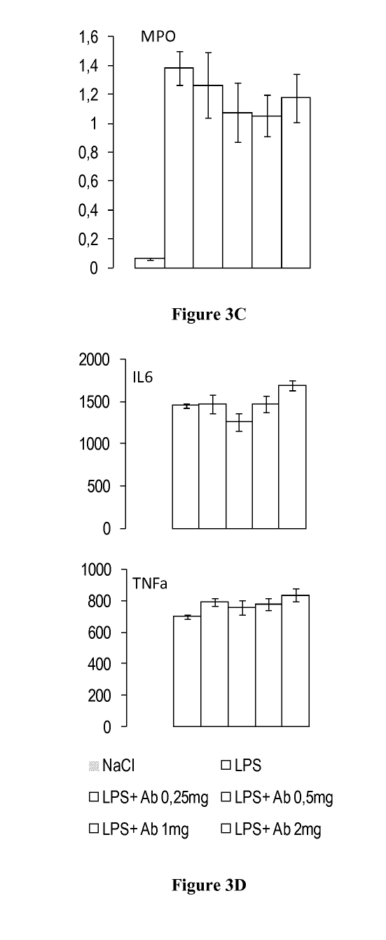

The inactivation of S100b reduced the lung response after LPS treatment without modifying the secretion of inflammatory signals. We then asked whether a neutralizing antibody directed against S100b could alter the AHR induced by the LPS. Using injection of a polyclonal anti-S100b antibody we performed a passive immunization in wild-type C57BL/6J (B6) mice. We compared three groups of B6 mice with 5 individuals: one treated with control saline solution, another positive control instillated with LPS and two additional ones with Intra-peritoneal injection i.p. of either 1 mg or 2 mg of polyclonal antibody against S100b, 16 h before receiving LPS (Vandal et al., 2003b). The experiment was carried out twice independently to reach n=10 individuals per group. The anti-S100b pre-injection lead to a significant decrease in the PenH values compared to mice which received only the LPS. 1 mg of the antibody was sufficient to reduce the AHR however the response was more homogenous with 2 mg (FIG. 3A). Anti-S100b had no effect on the inflammation observed in BALFs since none of the measured parameters were modified in mice which received 1 mg or 2 mg of antibody plus LPS compared to mice stimulated with LPS alone (FIG. 3B-E). In addition no abnormal behavioral changes were noticed in the treated mice. Similar results were observed in BALB/cJ mice treated with the neutralizing antibody in similar conditions (Data not shown). All the results obtained confirmed that S100b is involved is the AHR induced by LPS. The activity of S100b can be completely attenuated by a neutralizing antibody without strong consequence on the inflammation during LPS challenge.

Neutralizing Anti-S100b Reduced AHR in Prmt2^{+/-} Mice a Model of Exaggerated LPS Responses.

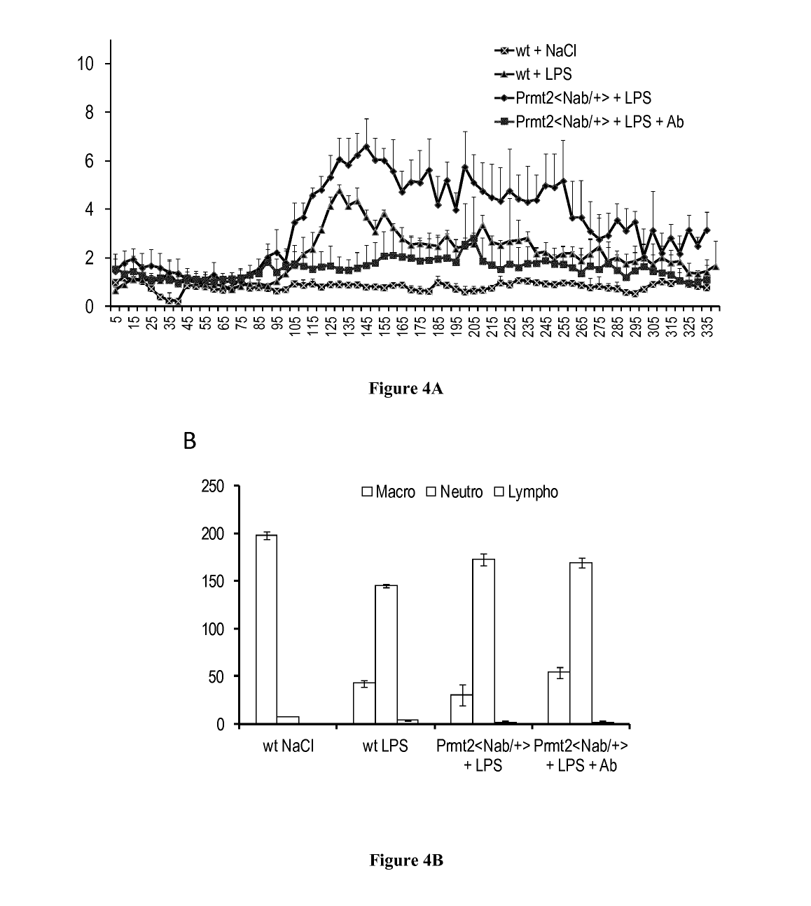

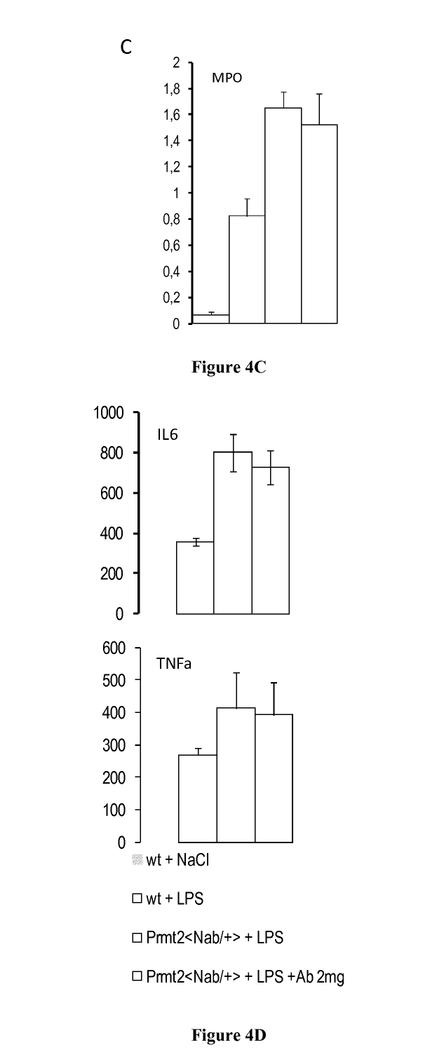

Prmt2^{+/-} mutant mice present a delayed but exacerbated AHR after LPS instillation compared to wt (Dalloneau et al., 2011a). These mice also present an increased pro-inflammatory response in both sera and BALFs. We then tested the action of the S100b antibody on the exaggerated airway response observed in Prmt2 deficient mice. The experiment was done on Prmt2^{+/-} mice as described in the paragraph before. The anti-S100B treatment lead to a significant decrease in the PenH values compared to mice which received only the LPS (FIG. 4A). The Prmt2^{+/-} mice treated with the S100B antibody displayed the same exacerbated inflammatory response with higher level of TNF-α and IL-6 secreted in BALFs similar to the Prmt2^{+/-} mice which received only the LPS, in comparison to wt mice (FIGS. 4B-E). Similar result was found for the Prmt2 homozygote mutants (data not shown).

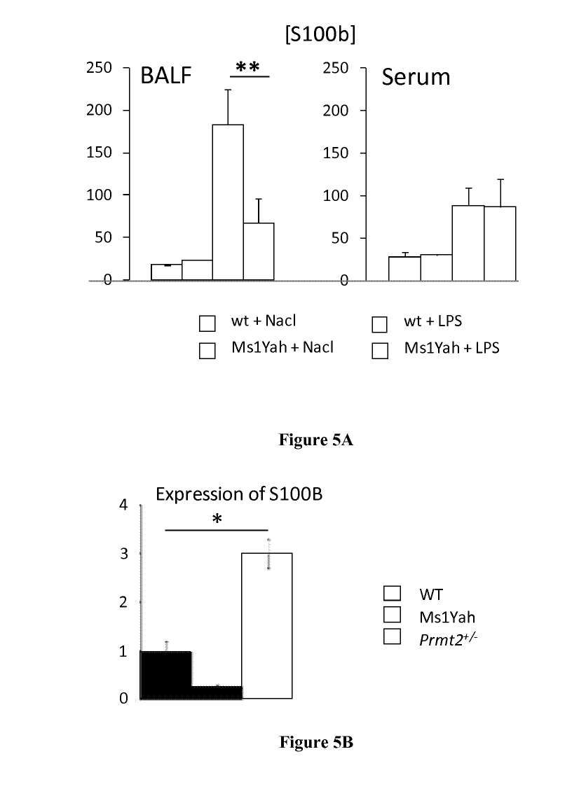

We then followed S100b response after systemic injection of LPS. S100b was found secreted more importantly in the lungs rather that in the serum (FIGS. 5 A,B). Interestingly, the expression of S100b was further stimulated by the loss of function of Prmt2 in mutant mice, reinforcing the hypothesis that S100b is key to the lung AHR induced by LPS (FIG. 5C). These results confirmed on one hand the involvement of S100b in the AHR induced by LPS, and on the other hand that S100b is not a major actor of the induction of the inflammatory response induced by LPS. It confirms (1) that S100b should act through a Prmt2 dependent pathway in the LPS-induced inflammatory response, the NF-Kb transcription factor that is controlled by the LPS/TLR4 and the S100b/AGER signaling pathway; and (2) that both inhibition of Prmt2 and S100b function is needed to recapitulate the LPS-induced phenotype in the Ms1Yah mutants (Besson et al., 2007).

Neutralizing Anti-S100b Reduced AHR in the OVA Mouse Model of Asthma

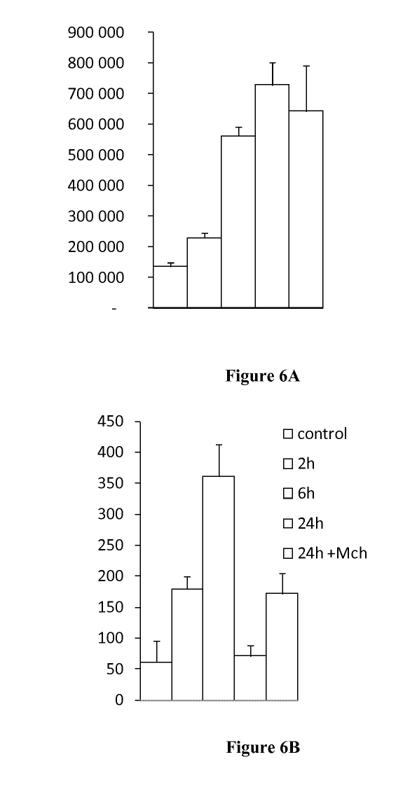

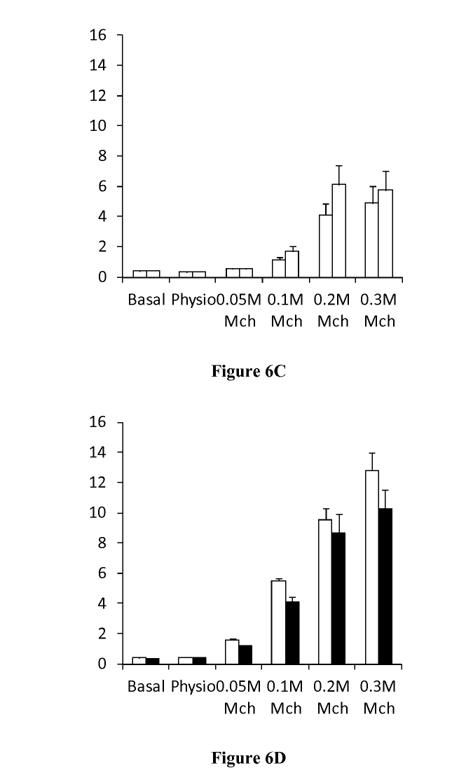

The S100b antibody reduced AHR induced by LPS in wt and Prmt2^{-/-} mice. Thus we explored the efficiency of S100b in another lung challenge, the ovalbumine (OVA) murine model of asthma. After sensitization with ovalbumin, BALB/cJ mice displayed an AHR and a tissues inflammation after further challenge with metacholine. Pulmonary allergic asthma requires the installation of an inflammatory response mediated by Lymphocytes T helpers 2 (type LTh2). These cells produce IL-4, triggering the recruitment of eosinophils to the site of the inflammation. We first checked the production of S100b in this OVA-induced asthma. Two groups of five mice were sensitized to ovalbumine at days 0 and 7 and challenged with the allergen on days 18, 19, 20 and 21. On day 22 one group received increased amount of metacholin (Mch, 0.05; 0.1; 0.2 and 0.3M). Mice were sacrificed at t=0, 2 h, 6 h and 24 h and BALFs and we evaluated the recruitment of eosinophils and the S100b production (FIG. 6). The eosinophils were recruited until 2 h and were still present in the BALFs 24 h after the last challenge. S100b was detected in the sera at 6 h but decrease at 24 h in the group without Mch. The metacholin appeared to amplify the response: the mice having challenged with Mch showed a concentration of S100b higher than that found in the mice without challenge. Then we checked the effect of the antibody against S100b in naïve mice and sensitized mice (FIG. 6C-D). Two groups of 6 mice were sensitized and then challenged to ovalbumine as described below, before measuring the respiratory function at day 22 with a Mch challenge.

One group received 2 mg of the S100B antibody on day 17, 16 h before the OVA challenge. The experiment was repeated twice independently. No significant effect was observed in control naïve mice which respond normally to the Mch challenge (FIG. 6C). After OVA sensitization, mouse displayed a hyperreactivity to Mch and we found that the more important the amount of Mch is, the more the effect of the antibody is marked (FIG. 6D). Indeed, mice which received the antibody tend to have lower values of PenH compared to control mice. The inhibition of the S100b protein decreased the Mch-induced hyperreactivity in the OVA model of asthma. These results let foresee the therapeutic impact of a molecule able to inhibit the activity of S100b, in particular in the lung diseases such as asthma.

S100b is Expressed in Lung and Increased after LPS Stimulation.

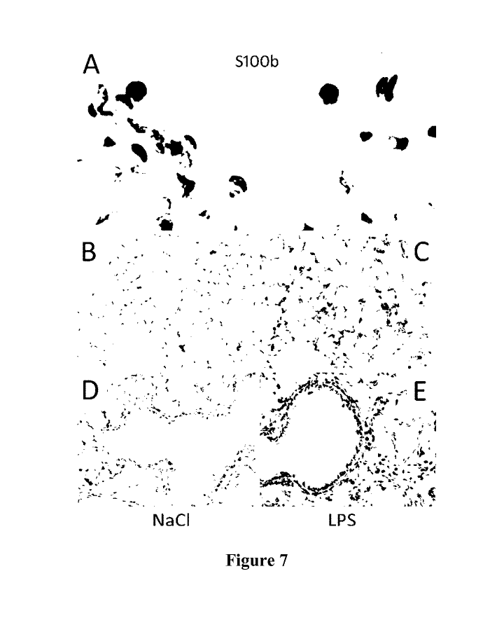

S100b-directed neutralizing antibody is able to reduce the AHR without modifying the LPS-induced inflammatory response. We then tried to decipher what are the cellular components of the S100b response? In order to detect S100b in the murine lung before and after LPS treatment, we performed immunohistochemistery on mouse lung tissue sections. In normal conditions with NaCl treatment, S100b was found scattered in cells of the alveolar walls. A few expressing cells were found in the inter-alveolar spaces and were identified as macrophages (FIG. 7A) based on their location and morphology. Four hours after LPS instillation, S100b expression was much higher in LPS-treated lungs compared to control NaCl-treated lungs (FIG. 7B-E). Hematoxylin & eosin (H&E) staining of lung sections after four hours of instillation with LPS revealed an increase in inflammatory cells, essentially neutrophils and macrophages expressing S100b, in the alveolar and peri-bronchiolar spaces of the lung. Using immunofluorescence, we found that S100b expressing cells were essentially found in interstitial spaces of the alveoli and around bronchioli similar as the inflammatory cells that were observed on H&E sections showing that S100b was expressed in the macrophages and neutrophils recruited after LPS instillation.

As LPS-mediated AHR was no more observed in anti-S100B treated mice, we checked the expression of S100B in the peri-bronchiolar smooth muscle cells and nervous plexus which are both involved in the response of the lung. Expression of S100b was found in nerve bundles underlying the bronchial epithelium and in the neuro-epithelial bodies (NEB) as shown by the double immunofluorescence stainings with the pan-neural marker, synaptic vesicular protein 2 (SV2) known to be expressed in the NEB and airway neural networks. S100b was not expressed in peri-bronchiolar smooth muscle cells as shown by the double immunofluorescence staining with a smooth muscle α-actin antibody. Overall partial expression of S100b was seen in the SV2 expressing nerves showing that S100b would be restricted to particular regions of the nerves.

Discussion:

In this report we demonstrated a new role of S100b in the regulation of the respiratory function in the LPS model of acute lung injury (ALI) and in the OVA model of asthma. S100b had a dose dependent effect on the regulation of the AHR induced by LPS without changes in key inflammatory parameters. The use of a neutralizing antibody against S100b allowed a significant reduction of the LPS-induced AHR in C57BL/6J, BALB/cJ and Prmt2 deficient mice (Dalloneau et al., 2011b). S100b was secreted in the BALF from the stimulated resident macrophages and the recruited neutrophils near the smooth muscle cells in the alveoli and at the bronchiole, suggesting a potential interference of S100b with the control of muscular contraction during AHR.

The interaction of S100b with the receptor AGER induces in vivo, the activation of the NF-κB transcription factor (Yan et al., 1994), and subsequently the production of IL-6, in particular in the liver and the lungs (Schmidt et al., 2000) as well as the expression of TNF-α (Hofmann et al., 1999). AGER is known to contribute to the inflammatory response either with an indirect or a direct interaction with LPS during the septic shock (Yamamoto et al., 2011) or in the lungs (Yamakawa et al., 2011). AGER has been proposed as a marker of lung injury (Su et al., 2009) and its expression was increased after LPS stimulation (Zhang et al., 2008). Crosstalks between the AGER and the TLR4 signaling cascade have been already described but for another ligand of AGER (Park et al., 2006; Qin et al., 2009). Here, we found a direct impact of S100b on the AHR in two lung challenges with no difference in the LPS-induced inflammatory response, in S100b mutant mice. No changes in cellular recruitments and in pro-inflammatory cytokines secretion were observed. The expression of IL-6 and TNF-a was not impacted suggesting that no modification of TLR4/NF-κB pathway even in the absence of S100b in mutant mice or using the neutralizing antibodies. We found that S100b expression was regulated by the LPS and even upregulated when Prmt2 an indirect inhibitor of NF-kb is inactivated (Dalloneau et al., 2011a). Such LPS dependent expression of S100b has been observed in astrocytes and microglia (Donato et al., 2009; Guerra et al., 2011). S100b signals through the AGER that shares common intermediates with the TLR4/TIRAP/MYD88/NF-kB pathway, contributing to the airway and inflammatory responses in the models of acute lung challenges (Sakaguchi et al., 2011a).

Trisomic and partial monosomic 21 patients present an increased sensitivity to respiratory infections which represent actually the main cause of death of the patient (Day et al., 2005; Pandit and Fitzgerald, 2012). To decipher this phenomenon, we developed different murine models of aneuploidy for regions of human chromosome 21 (Herault et al., 2012; Raveau et al., 2012). We previously demonstrated that the Ms1Yah model displayed an absence of AHR induced by LPS (Besson et al., 2007). Prmt2 was a gene deleted in the Ms1Yah model. It belongs to the protein arginine methyltransferase family and it regulates the nuclear accumulation of NF-kb. We showed that the expression of Prmt2 was dosage sensitive and decreased after LPS treatment (Besson et al., 2007; Dalloneau et al., 2011a). The study of the respiratory pattern of mice deficient for one or two copies of Prmt2 showed an exacerbated AHR which is also delayed and maintained in time compared to wt mice (Dalloneau et al., 2011a). This result pointed that at least another gene of the Prmt2-Col6a1 region is involved in the control of AHR observed in Ms1Yah mice. Then we focused our attention on S100b found expressed in lung (Besson et al., 2007). The present study of the respiratory pattern, after LPS instillation, showed a dosage effect of S100b on the regulation of the AHR. The PenH values tended to decrease in S100b^{+/-} mice and were significantly decreased in S100b^{-/-} mice. We demonstrated for the first time a clear role of S100b in LPS-induced AHR induced in a dose dependent manner. Similarly S100b was found increased in the OVA model of asthma and neutralizing antibodies can prevent part of the metacholine-induced constriction after sensitization and challenge. S100b was essentially known to be a marker of some types of cancer (Hwang et al., 2010), brain injury (\check{Z}urek and Fedora, 2011) and cardiac arrest (Song et al., 2010), but now S100b should be considered as a marker of lung response.

The loss of function S100b leads to an improvement of the respiratory function without modifying the inflammatory response in lung in mutant mice. Initially, we checked that the inhibition of S100b, in wild mice recapitulates the phenotype observed in S100b^{-/-} mice. We then used a blocking antibody to explore the function of S100b in respiratory function. The treatment with anti-S100B antibody reduced the HRA induced by the LPS without to modify the inflammatory response of the B6 mice. The same experiment was carried out in the Prmt2^{+/-} and Prmt2^{-/-} (data not shown) mice which present an exacerbated AHR. This model allowed us to validate the action of the antibody. Injection of the antibody in these mice, lead to a significant reduction in the PenH values, without changes in the inflammatory answer which remains higher in mutants.

S100b belongs to a family of 23 calcium binding protein and, contrary to other proteins of the same family, S100b is not a major actor of lung inflammation. In fact, S100a8 and S100a9 are secreted especially at sites of inflammation, where they induce chemotaxis and adhesion of neutrophils (Ryckman et al., 2003; Vandal et al., 2003a). S100a12 acts via interaction with AGER, resulting in the secretion of pro-inflammatory mediators (Hofmann et al., 1999). The serum concentrations of these S100 proteins correlate with inflammatory disease activity (Foell et al., 2004; Frosch et al., 2000). After LPS instillation, we demonstrated that the number of cells expressing S100b is increased but the inflammatory parameters measured in S100b deficient mice are not modified, suggesting that S100b is not necessary to induce inflammation. In normal human lung S100b is expressed in interstitial dendritic cells and peri-bronchial nerves at high level, in airway dendritic cells in smoke related damage and in interstitial dendritic cells in pneumonia (5). Here we found S100b expressed in alveolar resident macrophages, and in LPS-activated macrophages and recruited lung neutrophils. But even if the number of cells expressing S100b is increased during these lung diseases or challenges, the exact role of the protein is only revealed through this series of experiments showing that S100b is an actor of AHR and thus of respiratory diseases.

S100b is a calcium binding protein and as such it could participate to the control of muscle contraction. S100b is found in the cytoplasm of cells, and can stimulate Ca2+ fluxes, inhibite PKC-mediated phosphorylation and microtubule assembly. In the lungs, S100b is expressed in alveolar resident macrophages and recruited neutrophils after LPS stimulation, around the nerves epithelial bodies and in the nerves as previously described (5). No direct colocalization of S100b was observed with smooth muscle cells in lung. S100b is known as an intracellular and an extracellular regulator controlling the availability of calcium stock (18). If S100b act as a sense Ca2+ level, the secretion of S100b during LPS treatment may facilitate the muscular contraction, limiting the diffusion of calcium. Alternatively, S100b could change Ca2+ dependent intracellular signaling cascade such as the one involving the Ca2+/calmodulin-dependent protein phosphatase calcineurin, which upregulates numerous cytokines and proinflammatory factors in immune cells (Crabtree and Olson, 2002). Alternatively S100b is known to interact with several proteins from the cytoskeleton and thus could mediate cellular remodeling and cell migration (Donato et al., 2009). Further experiments will be needed to better understand the molecular and cellular mechanism of S100b in the AHR.

Reducing S100b function and decreasing AHR without altering key parameters of the inflammatory response represents an interesting path from a therapeutic point of view, in particular for long term treatment. Indeed, if we consider asthma seizure, the more handicapping aspect is the difficulty to breath of the patient, which results on the one hand to the reduction from the gauge of the bronchi following their contraction and on the other hand of the production of mucus. It is thus interesting, in this case, to facilitate the broncho-dilatation without modifying the inflammatory response in long term treatment. Indeed, treatment with neutralizing antibody could be used to reduce airway resistance observed in allergic asthma or in chronic obstructive broncho-pneumopathy. Today, the main treatment of the asthma remains of short duration. Long duration treatment with corticosteroid induces several side-effects such as hyperglycemia, osteoporosis, depression (Donihi et al., 2006). In the LPS model, the mice having received the antibody present relatively low values of PenH, and this lasting all the duration of acquisition. The mice (B6 and Prmt2^{+/-}) stimulated with the LPS alone present a rise in PenH values between 60 and 90 min, then after having reached a peak, these values find a basal level identical to the mice having received the antibody, 180 min after stimulation. This observation showed that the effect of the anti-S100b antibody is at least for 3 h in the LPS model; what is not astonishing since the half-life, in the serum, from the antibodies used in therapy for the autoimmune diseases varies between 8 days and 8 weeks (Chan and Carter, 2010). It will of course be necessary to check the effectiveness and duration of the action of the antibody in a murine model of asthma. All these results showed for the first time the importance of the S100b protein in the broncho-constriction and the use of the S100b antibody in the LPS murine model revealed the potential therapeutical effect of a molecule able to inhibit S100b.

### REFERENCES

Throughout this application, various references describe the state of the art to which this invention pertains. The disclosures of these references are hereby incorporated by reference into the present disclosure.
Arcuri, C. 2004. S100B Increases Proliferation in PC12 Neuronal Cells and Reduces Their Responsiveness to Nerve Growth Factor via Akt Activation. Journal of Biological Chemistry 280:4402-4414.
Besson, V., V. Brault, A. Duchon, D. Togbe, J. C. Bizot, V. F. Quesniaux, B. Ryffel, and Y. Herault. 2007. Modeling the monosomy for the telomeric part of human chromosome 21 reveals haploinsufficient genes modulating the inflammatory and airway responses. Hum Mol Genet 16:2040-2052.
Brett, J., A. M. Schmidt, S. D. Yan, Y. S. Zou, E. Weidman, D. Pinsky, R. Nowygrod, M. Neeper, C. Przysiecki, A. Shaw, A. Migheli, and D. Stern. 1993. Survey of the Distribution of a Newly Characterized Receptor for Advanced Glycation End Products in Tissues. American Journal of Pathology 143:1699-1712.
Chan, A. C., and P. J. Carter. 2010. Therapeutic antibodies for autoimmunity and inflammation. Nat Rev Immunol 10:301-316.
Cheng, C., K. Tsuneyama, R. Kominami, H. Shinohara, S. Sakurai, H. Yonekura, T. Watanabe, Y. Takano, H. Yamamoto, and Y. Yamamoto. 2005. Expression profiling of endogenous secretory receptor for advanced glycation end products in human organs. Modern Pathology 18:1385-1396.
Crabtree, G. R., and E. N. Olson. 2002. NFAT signaling: choreographing the social lives of cells. Cell 109 Suppl:S67-79.
Dahlin, K. 2004. Identification of Genes Differentially Expressed in Rat Alveolar Type I Cells. American Journal of Respiratory Cell and Molecular Biology 31:309-316.
Dalloneau, E., P. Lopes Pereira, V. Brault, E. G. Nabel, and Y. Herault. 2011a. Prmt2 Regulates the Lipopolysaccharide-Induced Responses in Lungs and Macrophages. The Journal of Immunology 187:4826-4834.
Dalloneau, E., P. L. Pereira, V. Brault, E. G. Nabel, and Y. Hérault. 2011b. Prmt2 regulates the lipopolysaccharide-induced responses in lungs and macrophages. J Immunol 187:4826-4834.
Day, S. M., D. J. Strauss, R. M. Shavelle, and R. J. Reynolds. 2005. Mortality and causes of death in persons with Down syndrome in California. Developmental Medicine & Child Neurology 47:171-176.
Donato. 2001. S100: a multigenic family of calcium-modulated proteins of the EF-hand type with intracellular and extracellular functional roles. Int J Biochem Cell Biol 33:637-668.
Donato, R. 1988. Calcium-independent, pH-regulated Effects of S-100 Proteins on Assembly-Disassembly of Brain Microtubule Protein in Vitro. the journal of biochemical chemistry 263:106-110.
Donato, R. 1999. Functional roles of S100 proteins, calcium-binding proteins of the EF-hand type. Biochimica et Biophysica Acta 1450:191-231.
Donato, R., G. Sorci, F. Riuzzi, C. Arcuri, R. Bianchi, F. Brozzi, C. Tubaro, and I. Giambanco. 2009. S100B's double life: Intracellular regulator and extracellular signal. Biochimica et Biophysica Acta (BBA)—Molecular Cell Research 1793:1008-1022.
Donihi, A. C., D. Raval, M. Saul, M. T. Korytkowski, and M. A. DeVita. 2006. Prevalence and predictors of corticosteroid-related hyperglycemia in hospitalized patients. Endocr Pract 12:358-362.
Eldik, L. J. V., and B. Zimmer. 1987. Secretion of S-IO0 from rat C6 glioma ceils. Brain Research 436:367-370.
Foell, D., H. Wittkowski, I. Hammerschmidt, N. Wulffraat, H. Schmeling, M. Frosch, G. Horneff, W. Kuis, C. Sorg, and J. Roth. 2004. Monitoring neutrophil activation in juvenile rheumatoid arthritis by S100A12 serum concentrations. ARTHRITIS & RHEUMATISM 50:1286-1295.
Frosch, M., A. Strey, T. Vogl, N. M. Wulffraat, W. Kuis, C. Sunderkötter, E. Harms, C. Sorg, and J. Roth. 2000. Myeloid-related proteins 8 and 14 are specifically secreted during interaction of phagocytes and activated endothelium and are useful markers for monitoring disease activity in pauciarticular-onset juvenile rheumatoid arthritis. Arthritis Rheum 43:628-637.
Guerra, M. C., L. S. Tortorelli, F. Galland, C. Da Ré, E. Negri, D. S. Engelke, L. Rodrigues, M. C. Leite, and C. A. Gonsalves. 2011. Lipopolysaccharide modulates astrocytic S100B secretion: a study in cerebrospinal fluid and astrocyte cultures from rats. J Neuroinflammation 8:128.
Herault, Y., A. Duchon, E. Velot, D. Maréchal, and V. Brault. 2012. The in vivo Down syndrome genomic library in mouse. Prog Brain Res 197:169-197.
Hofmann, M. A., S. Drury, C. Fu, W. Qu, A. Taguchi, Y. Lu, C. Avila, N. Kambham, A. Bierhaus, P. Nawroth, M. F. Neurath, T. Slattery, D. Beach, J. McClary, M. Nagashima, J. Morser, D. Stern, and A. M. Schmidt. 1999. RAGE Mediates a Novel Proinflammatory Axis: A Central Cell Surface Receptor for S100/Calgranulin Polypeptides. Cell 97:889-901.
Hwang, C.-C., H.-T. Chai, H.-W. Chen, H.-L. Tsai, C.-Y. Lu, F.-J. Yu, M.-Y. Huang, and J.-Y. Wang. 2010. S100B Protein Expressions as an Independent Predictor of Early Relapse in UICC Stages II and III Colon Cancer Patients after Curative Resection. Annals of Surgical Oncology 18:139-145.
Karin, M., and Y. Ben-Neriah. 2000. phosphorylation meets ubiquitination: The Control of NF-kB Activity. Annual Review of Immunology 18:621-663.
Matute-Bello, G., C. W. Frevert, and T. R. Martin. 2008. Animal models of acute lung injury. Am J Physiol Lung Cell Mol Physiol 295:L379-399.
Morbini, P., C. Villa, I. Campo, M. Zorzetto, S. Inghilleri, and M. Luisetti. 2006. The receptor for advanced glycation end products and its ligands: a new inflammatory pathway in lung disease? Modern Pathology
Nials, A. T., and S. Uddin. 2008. Mouse models of allergic asthma: acute and chronic allergen challenge. Dis Model Mech 1:213-220.
Pandit, C., and D. A. Fitzgerald. 2012. Respiratory problems in children with Down syndrome. J Paediatr Child Health 48:E147-152.
Park, J. S., F. Gamboni-Robertson, Q. He, D. Svetkauskaite, J. Y. Kim, D. Strassheim, J. W. Sohn, S. Yamada, I. Maruyama, A. Banerjee, A. Ishizaka, and E. Abraham. 2006. High mobility group box 1 protein interacts with multiple Toll-like receptors. Am J Physiol Cell Physiol 290:C917-924.
Qin, Y. H., S. M. Dai, G. S. Tang, J. Zhang, D. Ren, Z. W. Wang, and Q. Shen. 2009. HMGB1 enhances the proinflammatory activity of lipopolysaccharide by promoting the phosphorylation of MAPK p38 through receptor for advanced glycation end products. J Immunol 183:6244-6250.
Raveau, M., J. M. Lignon, V. Nalesso, A. Duchon, Y. Groner, A. J. Sharp, D. Dembele, V. Brault, and Y. Hérault. 2012. The app-runx1 region is critical for birth defects and electrocardiographic dysfunctions observed in a down syndrome mouse model. PLoS Genet. 8:e1002724.
Rustandi, R. R., D. M. Baldisseri, and D. J. Weber. 2000. Structure of the negative regulatory domain of p53 bound to S100B(ββ). nature structural biology 7:570-574.
Ryckman, C., K. Vandal, P. Rouleau, M. Talbot, and P. A. Tessier. 2003. Proinflammatory Activities of S100: Proteins S100A8, S100A9, and S100A8/A9 Induce Neutrophil Chemotaxis and Adhesion. The Journal of Immunology 170:3233-3242.
Sakaguchi, M., H. Murata, K. Yamamoto, T. Ono, Y. Sakaguchi, A. Motoyama, T. Hibino, K. Kataoka, and N. H. Huh. 2011a. TIRAP, an adaptor protein for TLR2/4, transduces a signal from RAGE phosphorylated upon ligand binding. PLoS One 6:e23132.
Sakaguchi, M., H. Murata, K. Yamamoto, T. Ono, Y. Sakaguchi, A. Motoyama, T. Hibino, K. Kataoka, and N. H. Huh. 2011b. TIRAP, an adaptor protein for TLR2/4, transduces a signal from RAGE phosphorylated upon ligand binding. PLoS One 6:e23132.
Sasaki, N., S. Toki, H. Chowei, T. Saito, N. Nakano, Y. Hayashi, M. Takeuchi, and Z. Makita. 2001. Immunohistochemical distribution of the receptor for advanced glycation end products in neurons and astrocytes in Alzheimer's disease. Brain Research 888:256-262.
Schmidt, A. M., S. D. Yan, S. F. Yan, and D. M. Stern. 2000. The biology of the receptor for advanced glycation end products and its ligands. Biochimica et Biophysica Acta (BBA)-Molecular Cell Research 1498:99-111.
Song, K. J., S. D. Shin, M. E. H. Ong, and J. S. Jeong. 2010. Can early serum levels of S100B protein predict the prognosis of patients with out-of-hospital cardiac arrest? Resuscitation 81:337-342.
Su, X., M. R. Looney, N. Gupta, and M. A. Matthay. 2009. Receptor for advanced glycation end-products (RAGE) is an indicator of direct lung injury in models of experimental lung injury. Am J Physiol Lung Cell Mol Physiol 297:L1-5.
Takeda, K., and S. Akira. 2007. Toll-like receptors. Curr Protoc Immunol Chapter 14:Unit 14.12.
Togbe, D., S. Schnyder-Candrian, B. Schnyder, E. Doz, N. Noulin, L. Janot, T. Secher, P. Gasse, C. Lima, F. R. Coelho, V. Vasseur, F. Erard, B. Ryffel, I. Couillin, and R. Moser. 2007. Toll-like receptor and tumour necrosis factor dependent endotoxin-induced acute lung injury. International Journal of Experimental Pathology 88:387-391.
Uchida, T. 2006. Receptor for Advanced Glycation End-Products Is a Marker of Type I Cell Injury in Acute Lung Injury. American Journal of Respiratory and Critical Care Medicine 173:1008-1015.
Umetsu, D. T., J. J. McIntire, O. Akbari, C. Macaubas, and R. H. DeKruyff. 2002. Asthma: an epidemic of dysregulated immunity. Nat Immunol 3:715-720.
Vandal, K., P. Rouleau, A. Boivin, C. Ryckman, M. Talbot, and P. A. Tessier. 2003a. Blockade of S100A8 and S100A9 suppresses neutrophil migration in response to lipopolysaccharide. J Immunol 171:2602-2609.
Vandal, K., P. Rouleau, A. Boivin, C. Ryckman, M. v. Talbot, and P. A. Tessier. 2003b. Blockade of S100A8 and S100A9 Suppresses Neutrophil Migration in Response to Lipopolysaccharide1. The Journal of Immunology 2602-2609.
Wilder, P. T., J. Lin, C. L. Bair, T. H. Charpentier, D. Yang, M. Liriano, K. M. Varney, A. Lee, A. B. Oppenheim, S. Adhya, F. Carrier, and D. J. Weber. 2006. Recognition of the tumor suppressor protein p53 and other protein targets by the calcium-binding protein S100B. Biochimica et Biophysica Acta (BBA)—Molecular Cell Research 1763:1284-1297.
Xiong, Z., D. O'Hanlon, L. E. Becker, J. Roder, J. F. MacDonald, and A. Marks. 2000. Enhanced calcium transients in glial cells in neonatal cerebellar cultures derived from S100B null mice. Exp Cell Res 257:281-289.
Yamakawa, N., T. Uchida, M. A. Matthay, and K. Makita. 2011. Proteolytic release of the receptor for advanced glycation end products from in vitro and in situ alveolar epithelial cells. Am J Physiol Lung Cell Mol Physiol 300:L516-525.
Yamamoto, Y., A. Harashima, H. Saito, K. Tsuneyama, S. Munesue, S. Motoyoshi, D. Han, T. Watanabe, M. Asano, S. Takasawa, H. Okamoto, S. Shimura, T. Karasawa, H. Yonekura, and H. Yamamoto. 2011. Septic shock is associated with receptor for advanced glycation end products ligation of LPS. J Immunol 186:3248-3257.
Yan, S. D., A. M. Schmidt, G. M. Anderson, J. Zhang, J. Bret, Y. S. Zou, D. Pinsky, and D. Stern. 1994. Enhanced Cellular Oxidant Stress by the Interaction of Advanced Glycation End Products with Their Receptors Binding Proteins. the journal of biochemical chemistry 269:9889-9897.
Yan, S. S., Z.-Y. Wu, H. P. Zhang, G. Furtado, X. Chen, S. F. Yan, A. M. Schmidt, C. Brown, A. Stern, J. LaFulle, L. Chess, D. M. Stern, and H. Jiang. 2003. Suppression of experimental autoimmune encephalomyelitis by selective blockade of encephalitogenic T-cell inflitration of the central nervous system. Nature Medicine 9:287-293.
Yoshimoto, T., M. Boehm, M. Olive, M. Crook, H. San, T. Langenickel, and E. Nabel. 2006. The arginine methyltransferase PRMT2 binds RB and regulates E2F function. Exp Cell Res 312:2040-2053.
Zhang, H., S. Tasaka, Y. Shiraishi, K. Fukunaga, W. Yamada, H. Seki, Y. Ogawa, K. Miyamoto, Y. Nakano, N. Hasegawa, T. Miyasho, I. Maruyama, and A. Ishizaka. 2008. Role of soluble receptor for advanced glycation end products on endotoxin-induced lung injury. Am J Respir Crit Care Med 178:356-362.
\check{Z}urek, J., and M. Fedora. 2011. The usefulness of S100B, NSE, GFAP, NF-H, secretagogin and Hsp70 as a predictive biomarker of outcome in children with traumatic brain injury. Acta Neurochirurgica

1. A method for reducing airway hyperresponse in a subject in need thereof comprising administering to the subject an agent selected from the group consisting of an anti-S100B antibody, an anti-S100B aptamer or an inhibitor of S100B gene expression.

2. The method according to claim 1 wherein the subject suffers from a airway disease selected from the group consisting of asthma, chronic obstructive pulmonary disease, allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma, sarcoid, reactive airway disease syndrome, interstitial lung disease, hyper-eosinophilic syndrome, rhinitis, sinusitis, exercise-induced asthma, pollution induced asthma and parasitic lung disease.

3. A method for determining whether a subject is at risk of having or developing an airway hyperresponse comprising
determining the level of S100B protein in a biological sample obtained from said subject.

4. The method of claim 3, wherein said step of determining comprises is performed by
contacting the biological sample with a binding partner that binds S100B with high affinity, thereby forming an S100B-binding partner complex, and
detecting the formation of the S100B-binding partner complex.

5. The method of claim 4, wherein said binding partner is an antibody or an aptamer.
